SEQUENCE LISTING (19 sequences)

SEQ ID NO: 1 - DNA, 21 bp, Artificial Sequence
  Sulfate Reducing Bacteria
ccngtncgyaccggtaaatgg

SEQ ID NO: 2 - DNA, 18 bp, Artificial Sequence
  Sulfate Reducing Bacteria
ccngtnggcgcntggttc

SEQ ID NO: 3 - DNA, 17 bp, Artificial Sequence
  Sulfate Reducing Bacteria
gagaacttgtgnccgga

SEQ ID NO: 4 - DNA, 15 bp, Artificial Sequence
  Sulfate Reducing Bacteria
cayacccagggytgg

SEQ ID NO: 5 - DNA, 14 bp, Artificial Sequence
  Sulfate Reducing Bacteria
cacacbaaggdtgg

SEQ ID NO: 6 - DNA, 15 bp, Artificial Sequence
  Sulfate Reducing Bacteria
cayacbcaaggctgg

SEQ ID NO: 7 - DNA, 15 bp, Artificial Sequence
  Sulfate Reducing Bacteria
catacdcaggghtgg

SEQ ID NO: 8 - DNA, 15 bp, Artificial Sequence
  Sulfate Reducing Bacteria
cacacdcagggrtgg

SEQ ID NO: 9 - DNA, 15 bp, Artificial Sequence
  Sulfate Reducing Bacteria
cacacdcagggytgg

SEQ ID NO: 10 - DNA, 15 bp, Artificial Sequence
  Sulfate Reducing Bacteria
catacccagggntay

SEQ ID NO: 11 - DNA, 15 bp, Artificial Sequence
  Sulfate Reducing Bacteria
catacwcaggghtat

SEQ ID NO: 12 - DNA, 21 bp, Artificial Sequence
  Sulfate Reducing Bacteria
ccatacnggrtaccakgcrcg

SEQ ID NO: 13 - DNA, 19 bp, Artificial Sequence
  Sulfate Reducing Bacteria
ggaagtcttcccangcttc

SEQ ID NO: 14 - DNA, 21 bp, Artificial Sequence
  Sulfate Reducing Bacteria
tgggaagabttcctvgacatg

SEQ ID NO: 15 - DNA, 17 bp, Artificial Sequence
  Sulfate Reducing Bacteria
gtgtarcagttrccrca

SEQ ID NO: 16 - DNA, 26 bp, Artificial Sequence
  Sulfate Reducing Bacteria
cagatcatgatcaayggtgartcyta

SEQ ID NO: 17 - DNA, 19 bp, Artificial Sequence
  Sulfate Reducing Bacteria
cgaagactactgngnvacc

SEQ ID NO: 18 - DNA, 21 bp, Artificial Sequence
  Sulfate Reducing Bacteria
cgcccacdccgtcngcvcagg

SEQ ID NO: 19 - DNA, 15 bp, Artificial Sequence
  Sulfate Reducing Bacteria
tsaacatgtgcggcg

(19) United States
(12) Patent Application Publication
(10) Pub. No.: US 2016/0289739 A1
(43) Pub. Date: Oct. 6, 2016

(54) PCR AMPLIFICATION METHODS AND KITS FOR DETECTING AND QUANTIFYING SULFATE-REDUCING BACTERIA

(71) Applicant: BAKER HUGHES INCORPORATED, Houston, TX (US)

(72) Inventors: Crystal Lee, Sugar Land, TX (US); Neil Sharma, Rockville, MD (US); Angela Reeves, Chicago, IL (US)

(73) Assignee: BAKER HUGHES INCORPORATED, Houston, TX (US)

(21) Appl. No.: 15/066,558

(22) Filed: Mar. 10, 2016

Related U.S. Application Data
(60) Provisional application No. 62/132,170, filed on Mar. 12, 2015.

(51) Int. Cl. C12Q 1/68

(52) U.S. Cl. CPC C12Q 1/686; C12Q 1/689

(57) ABSTRACT

A kit for optional use with a PCR method of amplification may include at least one reaction well, and an internal amplification control for a PCR amplification of an APS reductase gene having a sequence complementary to at least one sequence essentially identical to SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, and mixtures thereof. The kit may be used with a PCR method of amplifying at least one sulfur-reducing bacteria extracted from an oilfield fluid.

### TECHNICAL FIELD

The present invention relates to kits for use with PCR methods of amplifying, optionally detecting, and/or optionally quantifying sulfate-reducing bacteria.

### BACKGROUND

The presence of sulfate-reducing bacteria in many environments is undesirable, particularly in concentrations sufficient to cause significant corrosion of metals with aqueous solutions, including fresh and seawaters, having the sulfate-reducing bacteria (SRB) therein. SRBs are present in a variety of environments, including oil- and gas-bearing formations, soils, and wastewater. SRBs are also present in the gut of ruminant animals, particularly domestic animals (cattle) used as protein sources for human consumption.

Sulfate-reducing bacteria, such as members of the genera Desulfovibrio and Desulfotomaculum, may reduce sulfate and/or sulfite under suitable conditions (e.g. anaerobic conditions) and generate hydrogen sulfide, an odiferous, and poisonous gas. In addition, the sulfate-reducing bacteria may contact metals thereby causing corrosion to the metal, such as metal structures and conduits. “Sulfate-reducing bacteria” is defined herein to be bacteria capable of reducing sulfate to sulfite and/or bacteria capable of reducing sulfite to sulfide, regardless of the taxonomic group of the bacteria.

Traditionally, the monitoring of microbial populations has employed microbial growth tests where a sample is diluted to various levels and used to inoculate microbial growth media designed to favor the growth of various types of bacteria. After days to several weeks of incubation, the growth tests are scored based on the presence or absence of growth in these various microbiological media. Unfortunately, as numerous researchers show, only about 0.1% to about 10% bacteria from environmental samples can actually grow in an artificial medium, and a significant portion of bacteria growing in the media are not actually the target bacteria. Therefore, growth tests are unable to provide the accurate quantification of target bacteria in the samples. In addition, obtaining results from a serial dilution assay may take as long as three to four weeks.

To circumvent problems associated with such growth-based methods, many culture-independent genetic techniques have been developed in the past decade to detect pathogens in the field of medicine, the food industries, the oil and gas industries, and the like. Because many ecosystems have a relatively low abundance of microorganisms, the polymerase chain reaction (PCR) has been widely used to amplify the genetic signals of microbes in complex environmental samples. However, traditional PCR-based methods are significantly biased by amplification efficiency and the depletion of PCR reagents.

Real-time quantitative PCR (qPCR) may be used to detect and quantify a number of microorganisms. Quantitative PCR has also been used to determine the abundance of microorganisms in many different types of complex environmental samples, such as sediments, water, wastewater, and marine samples, qPCR may provide more accurate and reproducible quantification of microorganisms because qPCR quantifies the PCR products during the logarithmic phase of the reactions, which does not occur during traditional PCR methods. Moreover, qPCR offers a dynamic detection range of six orders of magnitude or more, does not need post-PCR manipulation, and has the capability of high throughput analysis.

Digital PCR (dPCR) may be used to directly quantify and clonally amplify nucleic acids including DNA, cDNA, and/or RNA. dPCR may be more precise method than PCR and/or qPCR. Traditional PCR carries out one reaction per single sample. dPCR may carry out a single reaction within a sample, but the sample may be separated into a large number of partitions, and the reaction may be individually carried out within each partition. The separation may allow for a more reliable collection and a more sensitive measurement of nucleic acid amounts within the sample. dPCR may be useful for studying variations in gene sequences, such as copy number variants, point mutations, and the like, and dPCR may be routinely used for clonal amplification of samples for “next-generation sequencing.”

It would be desirable to have a method of detecting and optionally quantifying sulfate-reducing bacteria within a sample that is cost-effective and may occur in real time.

### SUMMARY

There is provided, in one form, a kit having at least one reaction well, and an internal amplification control for a PCR amplification of an APS reductase gene having a sequence complementary to at least one sequence essentially identical to SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, and mixtures thereof. The kit may be used with a PCR method of amplifying at least one sulfur-reducing bacteria extracted from an oilfield fluid.

An alternative non-limiting embodiment of the kit for use with a PCR method of amplification may include at least one primer and a probe. The primer may have an essentially identical sequence selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, and mixtures thereof. The probe may be specific for a fragment of an alpha subunit of an APS gene. The kit may be used with a PCR method of amplifying at least one sulfur-reducing bacteria extracted from an oilfield fluid.

In another non-limiting embodiment, a PCR amplification method for amplifying at least one nucleic acid from at least one sulfur-reducing bacteria is provided. The sulfur-reducing bacteria may be extracted from an oilfield fluid. The method may include inserting at least one reaction well into a HUNTER PCR™ machine, and amplifying the at least one nucleic acid to form an amplification product. The reaction well may include at least one nucleic acid in the presence of at least one primer. The primer(s) may have or include an essentially identical sequence selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, and mixtures thereof.

The kits and PCR amplification methods may be useful for quickly detecting sulfur-reducing bacteria within a sample.

### BRIEF DESCRIPTION OF THE DRAWINGS

In order to more fully understand the drawings referred to in the detailed description, a brief description of each drawing is presented here:

FIGS. 1-11 (SEQ ID NO:1 through SEQ ID NO:11) represent the nucleotide sequences of a forward primer usable to detect sulfur-reducing bacteria;

FIGS. 12-15 (SEQ ID NO:12 through SEQ ID NO: 15) represent the nucleotide sequence of a reverse primer usable to detect sulfur-reducing bacteria;

FIGS. 16-19 (SEQ ID NO:16 through SEQ ID NO: 19) represent the nucleotide sequence of a probe usable to detect sulfur-reducing bacteria;

FIG. 20 represents a non-limiting example of a restriction map of a plasmid pCI BSR used as an internal control, obtained from a plasmid pUC19;

FIG. 21 depicts a non-limiting embodiment of a reaction apparatus having a plurality of reaction wells that may be included in the kit and/or used with the PCR amplification method disclosed; and

FIG. 22 depicts an individual non-limiting reaction well.

### DETAILED DESCRIPTION

It has been discovered that a polymerase chain reaction (PCR) amplification method may be used to amplify at least one nucleic acid of at least one sulfur-reducing bacteria (SRB) in the presence of at least one primer to form an amplification product. This method of amplification, optional detection and optional quantification of SRBs present in a particular sample is much quicker than previous methods of detecting SRBs. For example, the PCR amplification methods described below may occur in an amount of time less than about a 7 calendar days, alternatively less than 2 calendar days, or less than 24 hours in another non-limiting embodiment. In yet another non-limiting embodiment, the PCR amplification methods may occur in less than 8 hours.

In an alternative embodiment, the method of amplification, optional detection and optional quantification may occur in an amount of time less than about a 7 calendar days, alternatively less than 2 calendar days, or less than 24 hours in another non-limiting embodiment. In yet another non-limiting embodiment, the PCR amplification, optional detection and optional quantification methods may occur in less than 8 hours.

‘Amplification’ as defined herein refers to any in vitro method for increasing the number of copies of a nucleotide sequence with the use of a DNA polymerase, such as a PCR method of amplification in a non-limiting embodiment. PCR amplification methods may include from about 10 cycles independently to about 50 cycles of denaturization and synthesis of a DNA molecule.

Prior to amplifying the nucleic acid(s) of the SRBs, the nucleic acids must first be extracted from a sample. The sample may be in any form necessary to obtain the sulfur-reducing bacteria, such as a fluid sample containing the SRB, a ground-up version of a tissue where it would be beneficial to determine whether the SRB are present in the tissue, and the like. In an alternative embodiment, a surface and/or surface solids suspected of having SRB contamination may be swabbed, and the swab may be placed in a fluid to obtain the SRB fluid sample. Non-limiting examples of a sample may be a food product, an animal tissue, a human tissue, a water sample, a lab surface, a metal surface, a paper mill industry surface, a wastewater within a wastewater treatment facility, a sample from the paint industry, and combinations thereof.

The nucleic acid may be or include, DNA, RNA (e.g. mRNA), and combinations thereof. The nucleic acid(s) from the sulphate-reducing bacteria within the sample may be extracted from the sample prior to amplifying the nucleic acid(s). Such extraction techniques of the nucleic acids from the sample may be carried out by standard techniques, which are well known to persons skilled in the art.

A non-limiting example of an extraction technique may be or include using the QIAamp Tissue Kit (QIAGEN, Hilden, Germany), the MP Bio Soil DNA kit, and the like. DNA from the SRBs may be extracted from a sample using the QIAamp Tissue Kit by performing the following method:
Centrifuge 1 mL of sample for 30 minutes at 15,000 rpm, and then remove the supernatant.
Add 200 μL of INSTAGENE™ template (Bio-rad laboratories, Hercules, Calif.) (previously homogenized) to the pellet.
Vortex the mixture for about 30 minutes at 56° C.
Vortex the mixture for 8 minutes at 100° C.
Centrifuge the sample for about 2 minutes at 12,000 rpm.
Remove about 20 μL of the supernatant to directly use in a PCR reaction.

Another non-limiting example of an extraction technique may be or include using the MP Bio Soil DNA kit. The DNA from the SRBs may be extracted from a sample by performing the following method:
Add up to 500 mg of a soil sample to a Lysing Matrix E tube.
Add 978 μl sodium phosphate buffer to the sample in the lysing matrix E tube.
Add 122 μl MT Buffer (an alkaline solution with surfactant to lyse a cell) to the lysing matrix E tube.
Homogenize the mixture in a FASTPREP™ Instrument for 40 seconds at a speed setting of about 6.0.
Centrifuge the mixture at 14,000×g for 5-10 minutes to pellet the debris; the centrifugation may be extended to about 15 minutes to enhance elimination of excessive debris from large samples, or from cells with complex cell walls.
Transfer the supernatant to a clean 2.0 mL microcentrifuge tube.
Add 250 μl of a protein precipitation solution (PPS) to the microcentrifuge tube and shake the tube by hand about 10 times.
Centrifuge the microcentrifuge tube at 14,000×g for about 5 minutes to pellet the precipitate.
Transfer the supernatant to a clean 15 mL tube. A 2.0 mL microcentrifuge tube may be used at this step, but better mixing and DNA binding may occur in a larger tube.
Resuspend the binding matrix suspension (a solution of small silicon beads) and add 1.0 mL of the resuspended binding matrix suspension to the supernatant within the 15 mL tube.
Place the 15 mL tube on a rotator or invert the 15 mL tube by hand for about 2 minutes to allow binding of the DNA with the binding matrix.
Place the 15 mL tube on a rack for about 3 minutes to allow settling of the binding matrix.
Remove and discard 500 μL of the supernatant being careful to avoid the settled binding matrix.
Resuspend the settled binding matrix in the remaining amount of the supernatant and transfer approximately 600 μL of the mixture to a SPIN™ Filter and centrifuge at 14,000×g for 1 minute.
Empty the catch tube (the catch tube ‘catches’ the portion of the mixture that goes through the filter) and add the remaining mixture from the resuspension of the settled binding matrix within the supernatant, from the above step, to the SPIN™ Filter and centrifuge at 14,000×g for 1 minute.
Empty the catch tube again.
Add 500 μL prepared SEWS-M (a wash buffer that contains ethanol) and gently resuspend the remaining pellet using the force of the liquid from the pipette tip (ensure that ethanol has been added to the Concentrated SEWS-M).
Centrifuge the resuspended pellet in SEWS-M at 14,000×g for 1 minute.
Empty the catch tube and replace.
Without any addition of liquid, centrifuge the resuspended pellet in SEWS-M a second time at 14,000×g for 2 minutes to “dry” the matrix of residual wash solution.
Discard the catch tube and replace with a new, clean catch tube.
Air dry the SPIN™ Filter for about 5 minutes at room temperature (about 65° F. to about 80° F.).
Gently resuspend the Binding Matrix pellet (the portion above or on top of the SPIN filter) in 50-100 μl of DES (DNase/Pyrogen-Free Water). To avoid over-dilution of the purified DNA, use the smallest amount of DES required to resuspend the Binding Matrix pellet. Yields may be increased by incubation for 5 minutes at 55° C. in a heat block or water bath.
Centrifuge the resuspended Binding Matrix pellet in DES at 14,000×g for 1 minute to bring eluted DNA into a clean catch tube; discard the SPIN filter.
The remaining DNA may be amplified by PCR amplification techniques and other downstream applications; store at about a temperature ranging from about −20° C. to about 4° C. until the extracted nucleic acids are ready to be amplified.

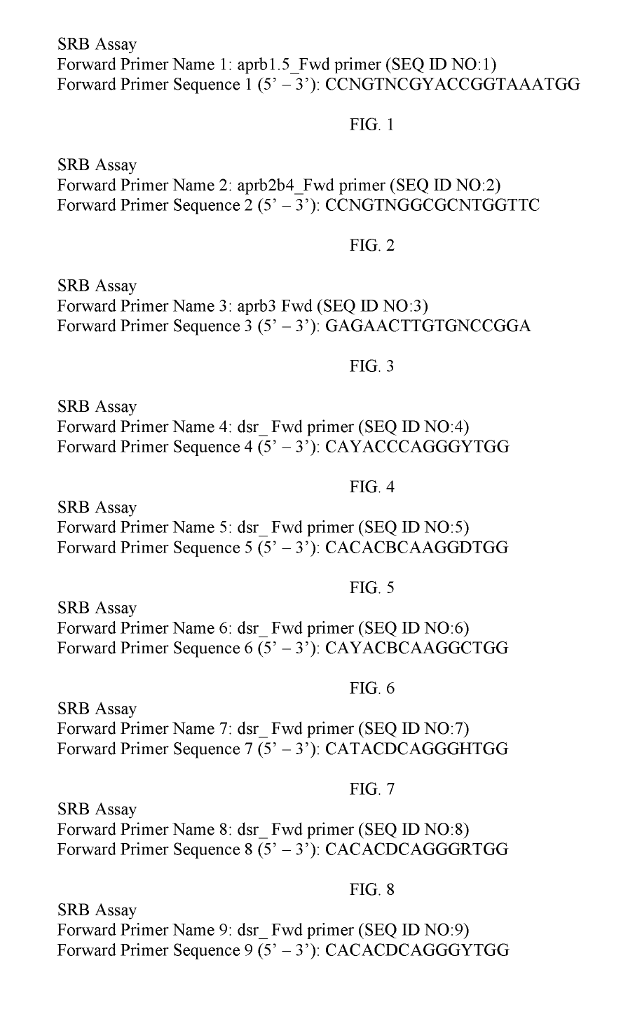

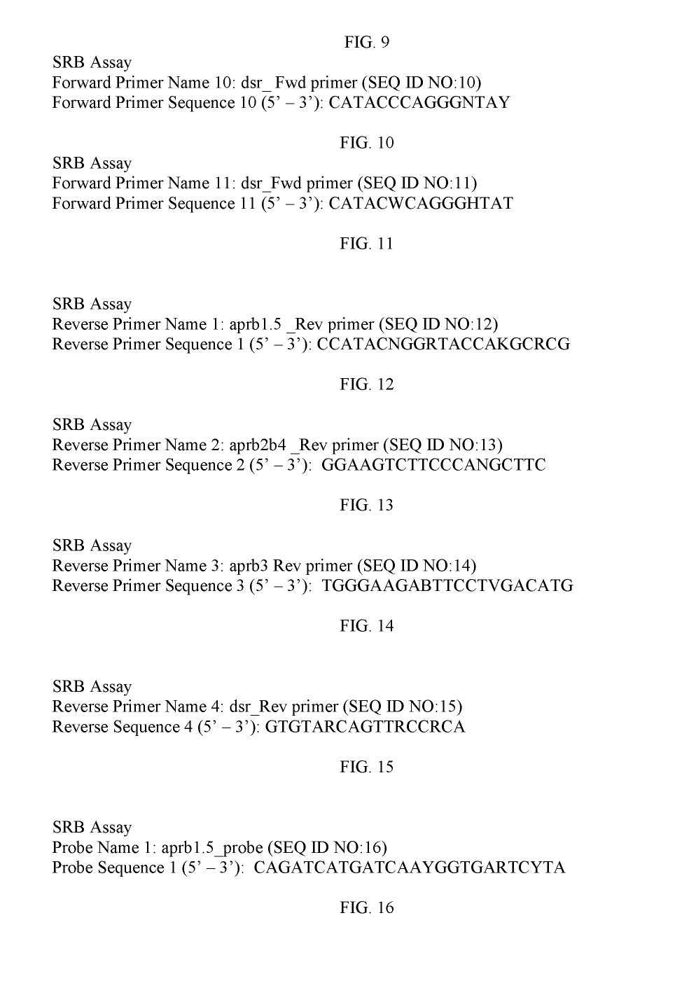

Once the nucleic acid(s) are extracted, the nucleic acid(s) may be combined with at least one primer in a reaction well to start and/or improve the amplification of the nucleic acids using a PCR method. The primer(s) may be or include a sequence that is essentially identical to SEQ ID NO:1 through SEQ ID: 15 (FIGS. 1-15), and mixtures thereof. Any of the sequences identified as SEQ ID NO:1 through SEQ ID NO: 11, or a combination thereof, may act as the forward primer. Any of the sequences identified as SEQ ID NO:12 through SEQ ID: 15, or a combination thereof, may act as the reverse primer. The primer(s) may be specific for amplification of at least a fragment of an alpha subunit of an APS reductase gene. Alternatively, the primer(s) may include an oligonucleotide from the alpha subunit of the APS reductase gene.

APS reductase (also known as Adenylylsulfate Reductase) allows the reduction of adenosine phosphosulfate (APS—a product of the activation of sulfate by ATP sulfurylase). APS reductase is a cytoplasmic enzyme containing two subunits (alpha and beta) known to be involved only in the anaerobic respiration of sulfate. This enzyme may not be present in non-sulfate-reducing organisms, since it is not involved in the assimilatory reduction that allows the incorporation of sulfur into various molecules necessary for life, such as amino acids and vitamins. Therefore, detecting fragments of the gene(s) that may code for APS reductase may allow for the detection of a sulfur-reducing bacteria.

“Essentially identical” is defined herein to mean that the sequence of the oligonucleotide is identical to at least one of the sequences (i.e. SEQ ID NO: 1 through SEQ ID NO:15), or that the oligonucleotide sequence differs from one of the sequences without affecting the capacity of these sequences to hybridize with the gene for the alpha subunit of APS reductase. A sequence that is “essentially identical” to SEQ ID NO:1 through SEQ ID NO:15 may differ therefrom by a substitution of one or more bases or by deletion of one or more bases located at the ends of the sequence, or alternatively by addition of one or more bases at the ends of the sequence.

‘Primer’ as defined herein refers to a single-stranded oligonucleotide that is extended by covalent bonding of nucleotide monomers during amplification or polymerization of a nucleic acid molecule. ‘Oligonucleotide’ as defined herein refers to a synthetic or natural molecule comprising a covalently linked sequence of nucleotides that are joined by a phosphodiester bond between the 3′ position of the pentose of one nucleotide and the 5′ position of the pentose of the adjacent nucleotide.

The components for a PCR method of amplification must be added to a reaction well prior to performing the PCR method of amplification. The reaction well may include or may be disposed within a reaction apparatus, such as but not limited to, a well plate, a cartridge apparatus, a test tube, and combinations thereof. The reaction apparatus may have or include only one reaction well, or the reaction well may have as many as 96 reactions wells, such as a standard 96 well plate known to those skilled in the art of performing PCR amplification methods.

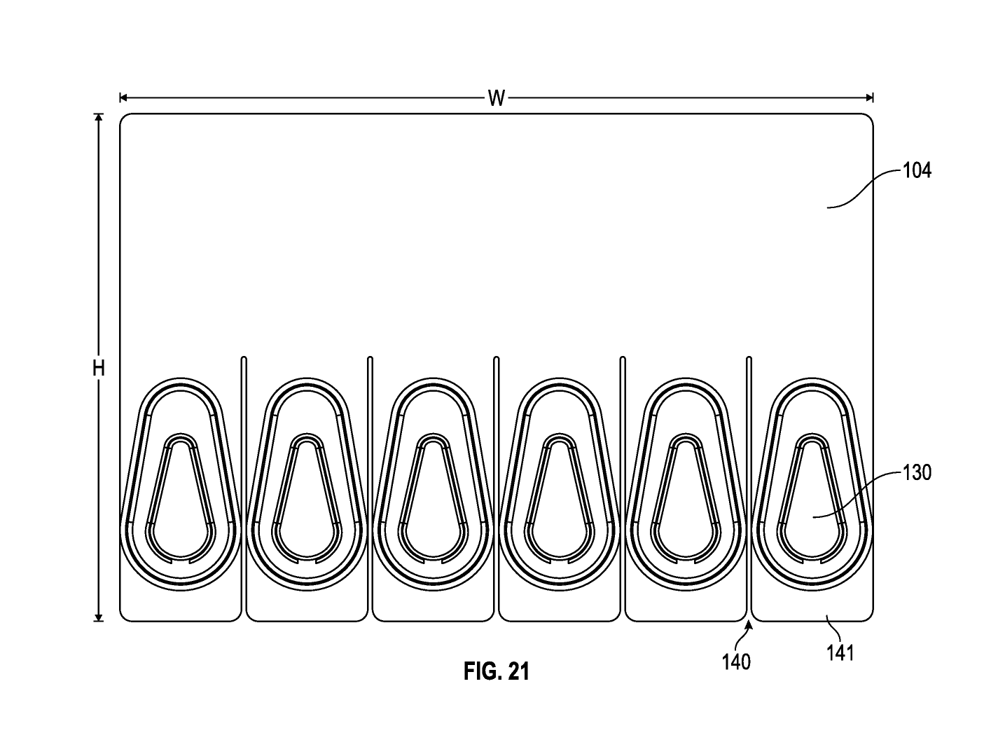

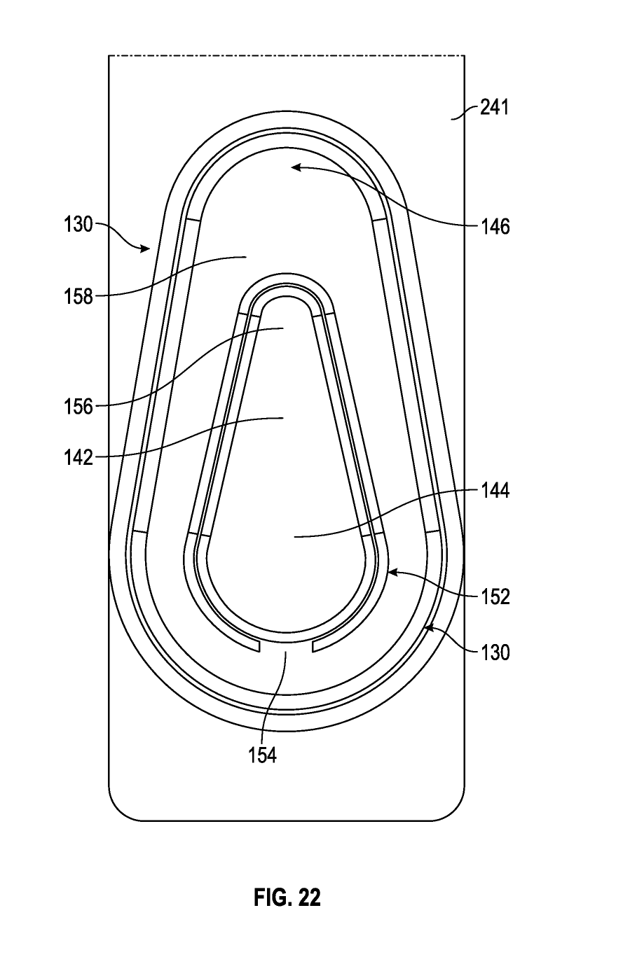

FIG. 21 depicts a non-limiting embodiment of a reaction well that may be included in the kit and/or used with the PCR amplification method disclosed. The reaction (also referred to as a cartridge) may include one or more reaction wells, such that multiple individual samples may be tested for a single analyte, or multiple analytes from a single sample may be tested. For example, a reaction apparatus 104 may run a sulfur-species panel that includes two or more types of sulfur-species bacteria on a single reaction apparatus 104. The reaction apparatus 104 may include a bar code to identify the specific assays therein. The bar code may be read by a bar code reader scanner of a PCR device (not shown) in a non-limiting embodiment to identify the test sample by a sample identification. The reaction apparatus 104 may be formed from a suitable material that is chemically compatible with reagents. In non-limiting embodiments, the reaction apparatus and/or reaction well(s) may be pre-loaded with an organism(s) to be tested, such as the list of sulfur-species bacteria mentioned herein. A non-limiting example of the reaction apparatus and/or reaction well is fully described in U.S. Patent Application No. 2012/0164649, which is herein incorporated by reference in its entirety.

The base of the reaction apparatus may have at least one slot 140 between each of the reaction wells 130. The slot(s) 140 may provide independent flexible fingers 141 to allow for individual seating of a reaction well 130 within the PCR device (not shown). An individual non-limiting reaction well 130 is depicted in FIG. 22, which may have an inner cavity portion 142 with a thermal interface wall 144. A top portion 146 of the reaction wells 130 provides a lead-in shape to provide a poke-yoke for insertion of a cover member (not shown), thus making cover insertion easier. For example, the lead-in area may have a width or outer diameter of, for example 5 mm. A top portion 156 of the inner cavity portion 142 may have a width or outer diameter, for example, of about 2 mm.

The thermal interface wall 144 may be configured to be the thermal interface between a reaction well 130 of the reaction apparatus 104 and a heat plate of a thermal cycler (not shown). The wall thickness of the thermal interface wall 144 may be, for example, 0.5 mm. The relatively large cross-sectional area of the inner cavity portion 142, and the relatively thin wall of the interface wall 144 may provide for high heat transfer from a thermal cycler to the sample volume. In addition, because of the flat aspect ratio of a non-limiting example of the reaction apparatus 104, the heat plate may be sized smaller and have a lower mass than in traditional PCR systems.

In a non-limiting embodiment, the reaction well(s) may be insertable into a HUNTER PCR™ machine in a vertical orientation in a non-limiting embodiment. The HUNTER PCR™ machine is fully described in U.S. Patent Application No. 2012/0164649, which is herein incorporated by reference in its entirety. Such orientation provides a side-view of the reaction wells by an optical scanning device within the PCR machine and allows for optical sensing to be performed in the lower portion of the PCR reaction well(s) in a single motion/pass across the reaction wells. The reaction wells, alone or within a reaction apparatus 104, may be inserted into the PCR machine such that the reaction well(s) are positioned adjacent a thermal cycler. In addition, the reaction apparatus may be configured to have as little thermal mass and thermal resistance as possible to further increase thermal cycling rates, as well as have a high thermal conductivity. The reaction well(s) may be mechanically compliant with a thermal cycler, such as by forming slits or slots between at least two reaction well(s).

In a non-limiting embodiment, the components may include the forward primer (also known as a sense primer), the reverse primer (also known as an antisense primer), PCR buffer, dNTP, DNA, Taq DNA polymerase, water, and combinations thereof. The amounts of the components within a reaction well are very well known to those skilled in the art, and the components within the reaction well may vary depending on the amounts of the other components present.

dNTPs are deoxynucleotide triphosphates included in a solution for purposes of PCR amplification. Stock dNTP solutions may have a pH of about 7, and the stability of dNTPs during repeated cycles of PCR may leave about 50% of the dNTPs remaining after about 50 PCR cycles. The concentration of each of the four dNTPs in solution ranges from about 20 μM to about 200 μM. Taq DNA polymerase is an enzyme used to replicate the DNA during the amplification where the enzyme may withstand the protein-denaturing conditions required for PCR methods of amplification.

PCR methods of amplification require particular conditions of temperature, reaction time, and optionally the presence of additional agents and/or reagents that are necessary for the fragment of the gene for the alpha subunit of APS reductase, to which the primers as defined above have hybridized, to be copied identically. Such conditions are well known to those skilled in the art. An average PCR program runs about 30 to about 65 cycles, but more or less cycles may be used depending on the conditions of the DNA, desired number of amplification products, time constraints, etc.

A non-limiting example of a PCR program having 42 total cycles may run where the first cycle runs for about 3 minutes at about 95 C, and cycles 2-6 run for about 1 minute at about 94° C. then 30 seconds at 54° C. then 10 seconds at 72° C. Cycles 7-41 may run for 30 seconds at 94° C., and then 10 seconds at 72° C. Cycle 42 may run for 5 minutes at 72 C and then held at 4 C until the reaction wells are removed from the PCR device. Computer processing may be used to analyze the crude amplification products. The PCR program mentioned above is strictly a non-limiting example and should not be deemed to limit the invention here.

An internal amplification control may be used in order to avoid an ambiguous interpretation of negative results of the PCR amplification method. For example, an absence of amplification by PCR may be due to problems of inhibition of the reaction, or to the absence of a target, i.e. the absence of DNA from the sulfur-reducing bacteria. The internal control may be a plasmid (FIG. 4) including oligonucleotide sequences that allows the amplification of a fragment of the APS reductase gene (289 base pairs) when no target is present in the sample. Thus, the presence of a fragment of 289 base pairs, without a fragment size having a different number of base pairs of the selected target, may indicate the functioning of the reaction and the absence of a specific target, i.e. the sulfate-reducing bacteria, from the sample. Also, the sequence intercalated between the primers αsp01 and αsp11 in the internal control differs by its size but also by its sequence (Leu2 gene), which makes it possible not to confuse the amplification of the internal control with the specific amplification of a fragment of the APS reductase gene whether the PCR analysis is performed on agarose gel or by hybridization. Such oligonucleotide sequences specific for a fragment of the APS reductase gene may be chosen in particular from SEQ ID NO:1 through SEQ ID NO: 15, and mixtures thereof.

When added in a limited concentration to the PCR reaction mixture, the plasmid allows the amplification of a DNA fragment when no specific target is present in the sample. This indicates the functioning of the reaction and the absence of a specific target, i.e. sulfate-reducing bacteria.

The amplification of at least one fragment of the APS reductase gene may allow for the detection of the fragment of the APS reductase gene, such as the gene for the alpha subunit of the APS reductase in a non-limiting embodiment. The gene amplification products may be optionally subjected to hybridization with a probe that is specific for a fragment of the gene for the alpha subunit of the APS reductase where the probe may be labeled in a detectable manner, such as but not limited to fluorescent labeling, radioactive labeling, chemiluminescent labeling, enzymatic labeling, and combinations thereof. ‘Gene’ is defined herein to mean a DNA sequence containing information required for expression of a polypeptide or protein.

Hybridizing the amplification product with a probe also requires particular conditions of temperature, reaction time, and preventing the hybridization of the oligonucleotide with sequences other than the gene for the alpha subunit of APS reductase. In a non-limiting example, the hybridization temperature may range from about 55° C. to about 65° C. The reaction time for the hybridization may range from about 0 seconds independently to about 60 seconds. The hybridization buffer may be a solution with a high ionic strength, such as a 6×SSC solution in a non-limiting example. As used herein with respect to a range, “independently” means that any threshold may be used together with another threshold to give a suitable alternative range.

The probe is a fragment of DNA used to detect the presence of nucleotide sequences that are complementary to the sequence in the probe. The probe hybridizes to a single-stranded nucleic acid, whose base sequence allows probe-target base pairing due to complementarity between the probe and the target (e.g. single-stranded DNA from the sulfur-reducing bacteria). First, the probe may be denatured (by heating or under alkaline conditions, such as exposure to sodium hydroxide) into single stranded DNA (ssDNA) and then hybridized to the target ssDNA, i.e. by Southern blotting in a non-limiting example. The hybridization may occur when the target ssDNA and probe are immobilized on a membrane (e.g. a gel) or in situ. ‘Target’ as used herein refers to DNA of the sulfur-reducing bacteria.

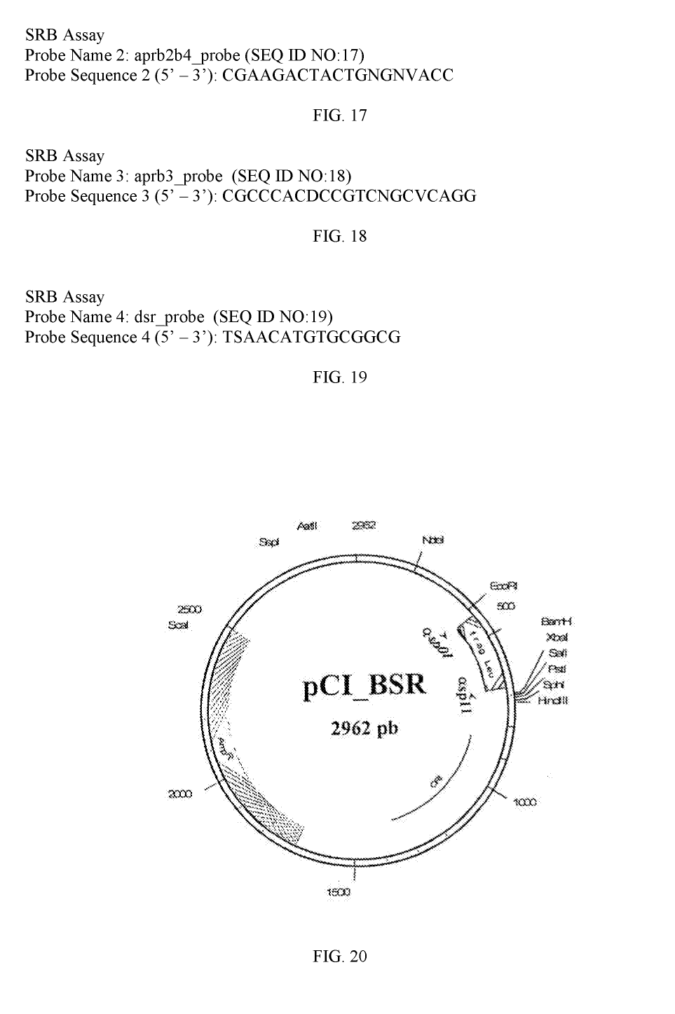

The resulting amplification product may be hybridized with a probe specific for a fragment of an alpha subunit of an APS gene. The probe may have a nucleotide sequence that specifically hybridizes to the complement of a nucleotide sequence essentially identical to at least one of SEQ ID NO: 16 through SEQ ID NO:19 (FIGS. 16-19). In a non-limiting embodiment, the probe may include a dye, such as those sold as QUASAR™ (e.g. QUASAR™ 670), BHQ PLUS, or combinations thereof.

A presence of hybridization and a degree of hybridization may be detected. The presence of hybridization may indicate the presence of the sulfate-reducing bacteria, and the degree of hybridization may enumerate the sulfate-reducing bacteria.

In a non-limiting embodiment, the method may be performed by
amplifying at least one nucleic acid of at least one sulfur-reducing bacteria in the presence of at least one primer to form an amplification product where the nucleic acid(s) are extracted from a sample prior to amplifying the nucleic acid(s). The primer(s) may include an oligonucleotide having a nucleotide sequence essentially identical to SEQ ID NO:1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO:12, SEQ ID NO:13, SEQ ID NO:14, SEQ ID NO:15, and mixtures thereof;
optionally hybridizing the amplification product with a probe having a nucleotide sequence that is essentially identical to SEQ ID NO: 16, SEQ ID NO: 17, SEQ ID NO: 18, SEQ ID NO: 19, and mixtures thereof; and
optionally detecting the hybridization complex formed between the product of amplification and the probe to indicate the presence of sulphate-reducing bacteria in the sample.

The type of sulfur-species bacteria that may be detected by the methods may be or include, but are not limited to, Desulfovibrio vulgaris, Desulfovibrio desuffuricans, Desulfovibrio aespoeensis, Thermodesulfobium narugense, Desulfotomaculum carboxydivorans, Desulfotomaculum ruminis, Desulfovibrio africanus, Desulfovibrio hydrothermalis, Desulfovibrio piezophilus, Desulfobacterium corrodens, Sulfate-reducing bacterium QLNR1, Desulfobacterium catecholicum, Desulfobacterium catecholicum, Desulfobulbus marinus, Desulfobulbus, Desulfobulbus propionicus, Desulfocapsa thiozymogenes, Desulfocapsa suffexigens, Desulforhopalus vacuolatus, Desulforhopalus, Desulfofustis glycolicus strain, Desulforhopalus singaporensis, Desulfobacterium, Desulfobacterium zeppelinii strain, Desulfobacterium autotrophicum, Desulfobacula phenolica, Desulfobacula toluolica Tol2, Sulfate-reducing bacterium JHA1, Desulfospira joergensenii, Desulfobacter, Desulfobacter postgatei, Desulfotignum, Desulfotignum balticum, Desulforegula conservatrix, Desulfocella, Desulfobotulus sapovorans, Desulfofrigus, Desulfonema magnum, Desulfonema limicola, Desulfobacterium indolicum, Desulfosarcina variabilis, Desulfatibacillum, Desulfococcus multivorans, Desulfococcus, Desulfonema ishimotonii, Desulfococcus oleovorans Hxd3, Desulfococcus niacini, Desulfotomaculum, Desulfotomaculum nigrificans, Desulfotomaculum ruminis, Desulfotomaculum halophilum, Desulfotomaculum acetoxidans, Desulfotomaculum gibsoniae, Desulfotomaculum sapomandens strain, Desulfotomaculum thermosapovorans, Desulfotomaculum, Desulfotomaculum geothermicum, Desulfotomaculum, Desulfosporosinus meridiei, Delta proteobacterium, Thermodesulforhabdus norvegica, Desulfacinum infernum, Desulfacinum hydrothermale, Desulforhabdus amnigena, Desulforhabdus, Desulforhabdus, Desulfomonile tiedjei, Desulfarculus baarsii, Sulfate-reducing bacterium, Sulfate-reducing bacterium, Sulfate-reducing bacterium, Desulfobacterium anilini, Delta proteobacterium, Desulfovibrio profundus strain, Desulfomicrobium baculatum, Desulfocaldus hobo, Desulfovibrio, Desulfovibrio piger, Desulfovibrio ferrophilus, Desulfonatronovibrio hydrogenovorans, Desulfovibrio, Desulfovibrio acrylicus, Desulfovibrio salexigens, Desulfovibrio oxyclinae, Desulfonauticus submarinus, Desulfothermus naphthae, Thermodesulfobacterium, Thermodesulfobacterium hveragerdense, Thermodesulfobacterium thermophilum, Thermodesulfatator indicus, Thermodesulfovibrio yellowstonii, Desulfosporosinus orientis, Desulfotomaculum thermobenzoicum, Desulfotomaculum, Desulfotomaculum, Desulfotomaculum solfataricum, Desulfotomaculum luciae strain, Desulfobacca acetoxidans, Desulfovibrio vulgaris, Desulfovibrio desulfuricans, Desulfovibrio alaskensis, Desulfovibrio magneticus, Desulfosporosinus acidiphilus, Desulfotomaculum kuznetsovii, Desulfotomaculum kuznetsovii, Desulfovibrio sulfodismutans, Desulfomicrobium baculatum, Desulfonatronum lacustre, Desulfohalobium retbaense, Desulfonauticus autotrophicus, Thermodesulfobacterium commune, Thermodesulfobacterium hveragerdense, Thermodesulfovibrio islandicus, Thermodesulfovibrio, Thermodesulfobacterium, Desulfotomaculum thermobenzoicum, Desulfotomaculum thermoacetoxidans, Desulfotomaculum thermocisternum, Desulfotomaculum australicum, Desulfotomaculum kuznetsovii, Desulfovibrio desulfuricans, Desulfovibrio alaskensis, Desulfovibrio vulgaris, Desulfovibrio salexigens, Desulfosporosinus acidiphilus, Desulfosporosinus meridiei, Desulfosporosinus orientis, Desulfotomaculum reducens, and combinations thereof.

In the foregoing specification, the invention has been described with reference to specific embodiments thereof, and has been described as effective in providing methods and compositions for PCR amplification methods, and primers and/or probes useful therefor. However, it will be evident that various modifications and changes can be made thereto without departing from the broader spirit or scope of the invention as set forth in the appended claims. Accordingly, the specification is to be regarded in an illustrative rather than a restrictive sense. For example, specific samples, nucleic acids, forward primers, reverse primers, probes, PCR cycles, sulfur-reducing bacteria, internal controls (plasmids), and the like falling within the claimed parameters, but not specifically identified or tried in a particular composition or method, are expected to be within the scope of this invention.

The present invention may suitably comprise, consist or consist essentially of the elements disclosed and may be practiced in the absence of an element not disclosed. For instance, the kit may consist of or consist essentially of at least one reaction well, and an internal amplification control for a PCR amplification of an APS reductase gene having a sequence complementary to at least one sequence essentially identical to SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, and mixtures thereof; the kit may be used with a PCR method of amplifying at least one sulfur-reducing bacteria extracted from an oilfield fluid.

The PCR amplification method for amplifying at least one nucleic acid from at least one sulfur-reducing bacteria may consist of or consist essentially of inserting at least one reaction well into a HUNTER PCR™ machine, and amplifying the nucleic acid(s) to form an amplification product; the reaction well may include at least one nucleic acid in the presence of at least one primer; the primer(s) may have or include an essentially identical sequence selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, and mixtures thereof.

The words “comprising” and “comprises” as used throughout the claims, are to be interpreted to mean “including but not limited to” and “includes but not limited to”, respectively.

What is claimed is:

1. A kit comprising:
at least one reaction well;
an internal amplification control for a PCR amplification of an APS reductase gene having a sequence complementary to at least one sequence essentially identical to SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, and mixtures thereof; and
wherein the kit is used with a PCR method of amplifying at least one sulfur-reducing bacteria extracted from an oilfield fluid.

2. The kit of claim 1 further comprising at least one agent selected from the group consisting of PCR buffer, at least one dNTP, Taq DNA polymerase, water, and combinations thereof.

3. The kit of claim 1, wherein the at least one reaction well is disposed within a cartridge apparatus configured to be disposed in a HUNTER™ PCR machine.

4. The kit of claim 1, wherein the at least one sulfur-species bacteria is selected from the group consisting of Desulfovibrio vulgaris, Desulfovibrio desulfuricans, Desulfovibrio aespoeensis, Thermodesulfobium narugense, Desulfotomaculum carboxydivorans, Desulfotomaculum ruminis, Desulfovibrio africanus, Desulfovibrio hydrothermalis, Desulfovibrio piezophilus, Desulfobacterium corrodens, Sulfate-reducing bacterium QLNR1, Desulfobacterium catecholicum, Desulfobacterium catecholicum, Desulfobulbus marinus, Desulfobulbus, Desulfobulbus propionicus, Desulfocapsa thiozymogenes, Desulfocapsa suffexigens, Desulforhopalus vacuolatus, Desulforhopalus, Desulfofustis glycolicus strain, Desulforhopalus singaporensis, Desulfobacterium, Desulfobacterium zeppelinii strain, Desulfobacterium autotrophicum, Desulfobacula phenolica, Desulfobacula toluolica Tol2, Sulfate-reducing bacterium JHA1, Desulfospira joergensenii, Desulfobacter, Desulfobacter postgatei, Desulfotignum, Desulfotignum balticum, Desulforegula conservatrix, Desulfocella, Desulfobotulus sapovorans, Desulfofrigus, Desulfonema magnum, Desulfonema limicola, Desulfobacterium indolicum, Desulfosarcina variabilis, Desulfatibacillum, Desulfococcus multivorans, Desulfococcus, Desulfonema ishimotonii, Desulfococcus oleovorans Hxd3, Desulfococcus niacini, Desulfotomaculum, Desulfotomaculum nigrificans, Desulfotomaculum ruminis, Desulfotomaculum halophilum, Desulfotomaculum acetoxidans, Desulfotomaculum gibsoniae, Desulfotomaculum sapomandens strain, Desulfotomaculum thermosapovorans, Desulfotomaculum, Desulfotomaculum geothermicum, Desulfotomaculum, Desulfosporosinus meridiei, Delta proteobacterium, Thermodesulforhabdus norvegica, Desulfacinum infernum, Desulfacinum hydrothermale, Desulforhabdus amnigena, Desulforhabdus, Desulforhabdus, Desulfomonile tiedjei, Desulfarculus baarsii, Sulfate-reducing bacterium, Sulfate-reducing bacterium, Sulfate-reducing bacterium, Desulfobacterium anilini, Delta proteobacterium, Desulfovibrio profundus strain, Desulfomicrobium baculatum, Desulfocaldus hobo, Desulfovibrio, Desulfovibrio piger, Desulfovibrio ferrophilus, Desulfonatronovibrio hydrogenovorans, Desulfovibrio, Desulfovibrio acrylicus, Desulfovibrio salexigens, Desulfovibrio oxyclinae, Desulfonauticus submarinus, Desulfothermus naphthae, Thermodesulfobacterium, Thermodesulfobacterium hveragerdense, Thermodesulfobacterium thermophilum, Thermodesulfatator indicus, Thermodesulfovibrio yellowstonii, Desulfosporosinus orientis, Desulfotomaculum thermobenzoicum, Desulfotomaculum, Desulfotomaculum, Desulfotomaculum solfataricum, Desulfotomaculum luciae strain, Desulfobacca acetoxidans, Desulfovibrio vulgaris, Desulfovibrio desulfuricans, Desulfovibrio alaskensis, Desulfovibrio magneticus, Desulfosporosinus acidiphilus, Desulfotomaculum kuznetsovii, Desulfotomaculum kuznetsovii, Desulfovibrio sulfodismutans, Desulfomicrobium baculatum, Desulfonatronum lacustre, Desulfohalobium retbaense, Desulfonauticus autotrophicus, Thermodesulfobacterium commune, Thermodesulfobacterium hveragerdense, Thermodesulfovibrio islandicus, Thermodesulfovibrio, Thermodesulfobacterium, Desulfotomaculum thermobenzoicum, Desulfotomaculum thermoacetoxidans, Desulfotomaculum thermocisternum, Desulfotomaculum australicum, Desulfotomaculum kuznetsovii, Desulfovibrio desulfuricans, Desulfovibrio alaskensis, Desulfovibrio vulgaris, Desulfovibrio salexigens, Desulfosporosinus acidiphilus, Desulfosporosinus meridiei, Desulfosporosinus orientis, Desulfotomaculum reducens, and combinations thereof.

5. The kit of claim 1, wherein the oilfield fluid is selected from the group consisting of oilfield water, a production fluid, a fracturing fluid, a drilling fluid, a completion fluid, a workover fluid, a packer fluid, a gas fluid, a crude oil, and mixtures thereof.

6. A kit for use with a PCR method of amplification comprising:
at least one primer comprising an essentially identical sequence selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, and mixtures thereof; and
a probe specific for a fragment of an alpha subunit of an APS gene; and
wherein the kit is used with a PCR method of amplifying at least one sulfur-reducing bacteria extracted from an oilfield fluid.

7. The kit of claim 6, wherein the probe has an essentially identical nucleotide sequence selected from the group consisting of SEQ ID NO: 16, SEQ ID NO: 17, SEQ ID NO: 18, SEQ ID NO: 19, and mixtures thereof.

8. The kit of claim 6, wherein the probe is detectably labeled.

9. The kit of claim 6, further comprising at least one nucleic acid of at least one sulfur-reducing bacteria.

10. The kit of claim 6, further comprising at least one agent selected from the group consisting of PCR buffer, dNTP, Taq DNA polymerase, water, and combinations thereof.

11. The kit of claim 6, further comprising an internal amplification control.

12. The kit of claim 6, further comprising at least one reaction well.

13. The kit of claim 12, wherein the reaction well is disposed within a reaction apparatus selected from the group consisting of a well plate, a cartridge apparatus, a test tube, and combinations thereof.

14. The kit of claim 6, wherein the at least one sulfur-species bacteria is selected from the group consisting of Desulfovibrio vulgaris, Desulfovibrio desulfuricans, Desulfovibrio aespoeensis, Thermodesulfobium narugense, Desulfotomaculum carboxydivorans, Desulfotomaculum ruminis, Desulfovibrio africanus, Desulfovibrio hydrothermalis, Desulfovibrio piezophilus, Desulfobacterium corrodens, Sulfate-reducing bacterium QLNR1, Desulfobacterium catecholicum, Desulfobacterium catecholicum, Desulfobulbus marinus, Desulfobulbus, Desulfobulbus propionicus, Desulfocapsa thiozymogenes, Desulfocapsa suffexigens, Desulforhopalus vacuolatus, Desulforhopalus, Desulfofustis glycolicus strain, Desulforhopalus singaporensis, Desulfobacterium, Desulfobacterium zeppelinii strain, Desulfobacterium autotrophicum, Desulfobacula phenolica, Desulfobacula toluolica Tol2, Sulfate-reducing bacterium JHA1, Desulfospira joergensenii, Desulfobacter, Desulfobacter postgatei, Desulfotignum, Desulfotignum balticum, Desulforegula conservatrix, Desulfocella, Desulfobotulus sapovorans, Desulfofrigus, Desulfonema magnum, Desulfonema limicola, Desulfobacterium indolicum, Desulfosarcina variabilis, Desulfatibacillum, Desulfococcus multivorans, Desulfococcus, Desulfonema ishimotonii, Desulfococcus oleovorans Hxd3, Desulfococcus niacini, Desulfotomaculum, Desulfotomaculum nigrificans, Desulfotomaculum ruminis, Desulfotomaculum halophilum, Desulfotomaculum acetoxidans, Desulfotomaculum gibsoniae, Desulfotomaculum sapomandens strain, Desulfotomaculum thermosapovorans, Desulfotomaculum, Desulfotomaculum geothermicum, Desulfotomaculum, Desulfosporosinus meridiei, Delta proteobacterium, Thermodesulforhabdus norvegica, Desulfacinum infernum, Desulfacinum hydrothermale, Desulforhabdus amnigena, Desulforhabdus, Desulforhabdus, Desulfomonile tiedjei, Desulfarculus baarsii, Sulfate-reducing bacterium, Sulfate-reducing bacterium, Sulfate-reducing bacterium, Desulfobacterium anilini, Delta proteobacterium, Desulfovibrio profundus strain, Desulfomicrobium baculatum, Desulfocaldus hobo, Desulfovibrio, Desulfovibrio piger, Desulfovibrio ferrophilus, Desulfonatronovibrio hydrogenovorans, Desulfovibrio, Desulfovibrio acrylicus, Desulfovibrio salexigens, Desulfovibrio oxyclinae, Desulfonauticus submarinus, Desulfothermus naphthae, Thermodesulfobacterium, Thermodesulfobacterium hveragerdense, Thermodesulfobacterium thermophilum, Thermodesulfatator indicus, Thermodesulfovibrio yellowstonii, Desulfosporosinus orientis, Desulfotomaculum thermobenzoicum, Desulfotomaculum, Desulfotomaculum, Desulfotomaculum solfataricum, Desulfotomaculum luciae strain, Desulfobacca acetoxidans, Desulfovibrio vulgaris, Desulfovibrio desulfuricans, Desulfovibrio alaskensis, Desulfovibrio magneticus, Desulfosporosinus acidiphilus, Desulfotomaculum kuznetsovii, Desulfotomaculum kuznetsovii, Desulfovibrio sulfodismutans, Desulfomicrobium baculatum, Desulfonatronum lacustre, Desulfohalobium retbaense, Desulfonauticus autotrophicus, Thermodesulfobacterium commune, Thermodesulfobacterium hveragerdense, Thermodesulfovibrio islandicus, Thermodesulfovibrio, Thermodesulfobacterium, Desulfotomaculum thermobenzoicum, Desulfotomaculum thermoacetoxidans, Desulfotomaculum thermocisternum, Desulfotomaculum australicum, Desulfotomaculum kuznetsovii, Desulfovibrio desulfuricans, Desulfovibrio alaskensis, Desulfovibrio vulgaris, Desulfovibrio salexigens, Desulfosporosinus acidiphilus, Desulfosporosinus meridiei, Desulfosporosinus orientis, Desulfotomaculum reducens, and combinations thereof.

15. The kit of claim 6, wherein the oilfield fluid is selected from the group consisting of oilfield water, a production fluid, a fracturing fluid, a drilling fluid, a completion fluid, a workover fluid, a packer fluid, a gas fluid, a crude oil, and mixtures thereof.

16. A PCR amplification method for amplifying at least one nucleic acid from at least one sulfur-reducing bacteria; wherein the at least one sulfur-reducing bacteria is extracted from an oilfield fluid; wherein the method comprises:
inserting at least one reaction well into a HUNTER PCR™ machine; wherein the at least one reaction well comprises the at least one nucleic acid in the presence of at least one primer; wherein the at least one primer comprises an essentially identical sequence selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, and mixtures thereof; and
amplifying the at least one nucleic acid to form an amplification product.

17. The method of claim 16, wherein the oilfield fluid is selected from the group consisting of oilfield water, a production fluid, a fracturing fluid, a drilling fluid, a completion fluid, a workover fluid, a packer fluid, a gas fluid, a crude oil, and mixtures thereof.

18. The method of claim 16, wherein the at least one sulfur-species bacterium is selected from the group consisting of Desulfovibrio vulgaris, Desulfovibrio desulfuricans, Desulfovibrio aespoeensis, Thermodesulfobium narugense, Desulfotomaculum carboxydivorans, Desulfotomaculum ruminis, Desulfovibrio africanus, Desulfovibrio hydrothermalis, Desulfovibrio piezophilus, Desulfobacterium corrodens, Sulfate-reducing bacterium QLNR1, Desulfobacterium catecholicum, Desulfobacterium catecholicum, Desulfobulbus marinus, Desulfobulbus, Desulfobulbus propionicus, Desulfocapsa thiozymogenes, Desulfocapsa sulfexigens, Desulforhopalus vacuolatus, Desulforhopalus, Desulfofustis glycolicus strain, Desulforhopalus singaporensis, Desulfobacterium, Desulfobacterium zeppelinii strain, Desulfobacterium autotrophicum, Desulfobacula phenolica, Desulfobacula toluolica Tol2, Sulfate-reducing bacterium JHA1, Desulfospira joergensenii, Desulfobacter, Desulfobacter postgatei, Desulfotignum, Desulfotignum balticum, Desulforegula conservatrix, Desulfocella, Desulfobotulus sapovorans, Desulfofrigus, Desulfonema magnum, Desulfonema limicola, Desulfobacterium indolicum, Desulfosarcina variabilis, Desulfatibacillum, Desulfococcus multivorans, Desulfococcus, Desulfonema ishimotonii, Desulfococcus oleovorans Hxd3, Desulfococcus niacini, Desulfotomaculum, Desulfotomaculum nigrificans, Desulfotomaculum ruminis, Desulfotomaculum halophilum, Desulfotomaculum acetoxidans, Desulfotomaculum gibsoniae, Desulfotomaculum sapomandens strain, Desulfotomaculum thermosapovorans, Desulfotomaculum, Desulfotomaculum geothermicum, Desulfotomaculum, Desulfosporosinus meridiei, Delta proteobacterium, Thermodesulforhabdus norvegica, Desulfacinum infernum, Desulfacinum hydrothermale, Desulforhabdus amnigena, Desulforhabdus, Desulforhabdus, Desulfomonile tiedjei, Desulfarculus baarsii, Sulfate-reducing bacterium, Sulfate-reducing bacterium, Sulfate-reducing bacterium, Desulfobacterium anilini, Delta proteobacterium, Desulfovibrio profundus strain, Desulfomicrobium baculatum, Desulfocaldus hobo, Desulfovibrio, Desulfovibrio piger, Desulfovibrio ferrophilus, Desulfonatronovibrio hydrogenovorans, Desulfovibrio, Desulfovibrio acrylicus, Desulfovibrio salexigens, Desulfovibrio oxyclinae, Desulfonauticus submarinus, Desulfothermus naphthae, Thermodesulfobacterium, Thermodesulfobacterium hveragerdense, Thermodesulfobacterium thermophilum, Thermodesulfatator indicus, Thermodesulfovibrio yellowstonii, Desulfosporosinus orientis, Desulfotomaculum thermobenzoicum, Desulfotomaculum, Desulfotomaculum, Desulfotomaculum solfataricum, Desulfotomaculum luciae strain, Desulfobacca acetoxidans, Desulfovibrio vulgaris, Desulfovibrio desuffuricans, Desulfovibrio alaskensis, Desulfovibrio magneticus, Desulfosporosinus acidiphilus, Desulfotomaculum kuznetsovii, Desulfotomaculum kuznetsovii, Desulfovibrio sulfodismutans, Desulfomicrobium baculatum, Desulfonatronum lacustre, Desulfohalobium retbaense, Desulfonauticus autotrophicus, Thermodesulfobacterium commune, Thermodesulfobacterium hveragerdense, Thermodesulfovibrio islandicus, Thermodesulfovibrio, Thermodesulfobacterium, Desulfotomaculum thermobenzoicum, Desulfotomaculum thermoacetoxidans, Desulfotomaculum thermocisternum, Desulfotomaculum australicum, Desulfotomaculum kuznetsovii, Desulfovibrio desuffuricans, Desulfovibrio alaskensis, Desulfovibrio vulgaris, Desulfovibrio salexigens, Desulfosporosinus acidiphilus, Desulfosporosinus meridiei, Desulfosporosinus orientis, Desulfotomaculum reducens, and combinations thereof.

19. The method of claim 16 further comprising detecting a presence of the at least one sulfur-reducing bacteria in the oilfield fluid.

20. The method of claim 16, wherein the at least one primer is specific for amplification of at least a fragment of an alpha subunit of an APS reductase gene.
